(19) United States
(12) Patent Application Publication
(10) Pub. No.: US 2025/0204749 A1
(43) Pub. Date: Jun. 26, 2025

(54) VARIABLE STIFFNESS MEDICAL DEVICE SHAFT

(71) Applicant: Verathon Inc., Bothell, WA (US)

(72) Inventors: Lauren Willeson, Burnaby (CA); Gary Leong, Burnaby (CA)

(73) Assignee: Verathon Inc., Bothell, WA (US)

(21) Appl. No.: 18/990,834

(22) Filed: Dec. 20, 2024

Related U.S. Application Data
(60) Provisional application No. 63/613,657, filed on Dec. 21, 2023.

(51) Int. Cl. A61B 1/00

(52) U.S. Cl. CPC A61B 1/00078

(57) ABSTRACT

In some embodiments, a system may include a shaft that defines a void and has a length extending between a proximal end and a distal end. The system may also include an elongated member disposed within the void of the shaft and along at least a portion of the length of the shaft. The elongated member may be configured to define at least two regions. The at least two regions may each have a different stiffness, facilitating a variable stiffness along the length of the shaft. Other systems having an elongated members providing variable stiffness along the length of a shaft are disclosed.

### CROSS-REFERENCE TO RELATED APPLICATIONS

This applications claims the benefit of priority under 35 U.S.C. § 119(e) to prior U.S. Provisional Patent Application No. 63/613,657 filed on Dec. 21, 2023, the disclosure of which is incorporated by reference herein in its entirety.

### FIELD OF DISCLOSURE

The disclosed systems are related to the field of medical devices used in medical procedures and diagnostics. More particularly, the disclosed systems are concerned with variable stiffness shafts of the medical devices.

### BACKGROUND

Medical devices, such as endoscopes, catheters, etc., have shafts that are configured to be inserted into a patient's anatomy. Depending on the medical procedure and particular anatomy of a patient, the shaft may be required to flex in order to reach a particular portion of a patient's anatomy. Conventional medical device shafts that are rigid throughout the length of the shaft are hard to navigate through a patient's anatomy due to the lack of flexibility. Conversely, conventional medical device shafts that are flexible throughout the length of the shaft also presents issues when trying to insert and navigate the flexible shaft through a patient's anatomy due to the lack of push-ability of the shaft into the patient. Some medical devices utilize a shaft with varying stiffness throughout the length of the shaft so as to allow a flexible distal end for navigating through a patient's anatomy and a rigid proximal end for improved push-ability. However, these conventional varying stiffness shafts require significant design changes to the shaft, such as varying the material of the shaft or shaft components along the length of the shaft.

### SUMMARY

In some embodiments, a system may include a shaft that defines a void and has a length extending between a proximal end and a distal end. The system may also include an elongated member disposed within the void of the shaft and along at least a portion of the length of the shaft. The elongated member may be configured to define at least two regions. The at least two regions may each have a different stiffness, facilitating a variable stiffness along the length of the shaft.

In some embodiments, the void of the shaft may include one or more lumens along the length of the shaft. In some embodiments, the elongated member may be disposed in one of the one or more lumens. In some embodiments, the elongated member may include a wire that wraps back around at an end of the wire such that there are two lengths of wire within a first region of the at least two regions and one length of wire within a second region of the at least two regions. In some embodiments, the shaft may define a first zone, a second zone, and a third zone. The first region of the wire may be disposed within the first zone, the second region of the wire may be disposed within the second zone, and the third zone may not include any length of the wire. In some embodiments, the end of the wire that wraps back around is disposed at an angle from a longitudinal axis of the shaft.

In some embodiments, the elongated member may include a first wire and a second wire. The first wire may be shorter than the second wire. In some embodiments, the first wire and the second wire may be disposed in a first region of the at least two regions and the second wire may be disposed in a second region of the at least two regions. In some embodiments, the shaft may define a first zone, a second zone, and a third zone. The first region may be disposed within the first zone, the second region may be disposed within the second zone, and the third zone may not include any length of the first wire or the second wire. In some embodiments, the first wire may have a first size cross-section and the second wire may have a second size cross-section. In some embodiments, the first wire and the second wire may each extend between a first end a second end. The first ends of the first wire and the second wire may each define an angle and the second ends of the first wire and the second wire may each define an end feature.

In some embodiments, a system may include a shaft that defines a void and has a length extending between a proximal end and a distal end. The elongated member may be disposed within the void of the shaft and along at least a portion of the length of the shaft. The elongated member may have a first diameter that tapers to at least a second diameter along a length of the elongated member, facilitating a variable stiffness along the length of the shaft.

In some embodiments, the void of the shaft may include one or more lumens along the length of the shaft. In some embodiments, the elongated member may be disposed in one of the one or more lumens. In some embodiments, the elongated member may include one or more wires. In some embodiments, the shaft may define a first zone, a second zone, and a third zone. The elongated member may taper along the length of the elongated member such that the first zone is stiffer than the second zone and the third zone.

In some embodiments, a system may include a shaft that defines a void and has a length extending between a proximal end and a distal end. The system may also include a plurality of removable rods disposed within the void of the shaft. A first portion of the plurality of removable rods may be disposed at a length that is shorter than a second portion of the plurality of removable rods, facilitating a variable stiffness along the length of the shaft.

In some embodiments, the void of the shaft may include one or more lumens along the length of the shaft. In some embodiments, the plurality of removable rods may be disposed in one of the one or more lumens. In some embodiments, the shaft may define a first zone, a second zone, and a third zone. The first portion and the second portion of the plurality of removable rods may be disposed within the first zone, the second portion of the plurality of removable rods may be disposed within the second zone, and the third zone of the shaft may not include any of the plurality of removable rods.

### BRIEF DESCRIPTION OF THE DRAWINGS

The features and advantages of the present disclosure will be more fully disclosed in, or rendered obvious by, the following detailed descriptions of example embodiments. The detailed descriptions of the example embodiments are to be considered together with the accompanying drawings wherein like numbers refer to like parts and further wherein:

FIG. 1 illustrates an isometric view of medical device shaft in accordance with some embodiments;

FIG. 2 illustrates an isometric view of a first example of an elongated member of a medical device in accordance with some embodiments;

FIG. 3A illustrates a side view of a second example of an elongated member of a medical device in accordance with some embodiments;

FIG. 3B illustrates a side view of an end of a second example of the elongated member of a medical device in accordance with some embodiments;

FIG. 4A illustrates an isometric view of a third example of an elongated member of a medical device in accordance with some embodiments;

FIG. 4B illustrates a side view of a third example of an elongated member of a medical device in accordance with some embodiments;

FIG. 4C illustrates a top down view of a third example of an elongated member of a medical device in accordance with some embodiments;

FIG. 5A illustrates an isometric view of a fourth example of an elongated member of a medical device in accordance with some embodiments;

FIG. 5B illustrates a side view of an end of a fourth example of the elongated member of a medical device in accordance with some embodiments;

FIG. 6A illustrates a side view of a fifth example of an elongated member of a medical device in accordance with some embodiments;

FIG. 6B illustrates a side view of an end of a fifth example of the elongated member of a medical device in accordance with some embodiments; and

FIG. 7 illustrates a cross sectional view of a shaft having a sixth example of an elongated member of a medical device in accordance with some embodiments.

While the present disclosure is susceptible to various modifications and alternative forms, specific embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the disclosure as defined by the appended claims.

### DETAILED DESCRIPTION

This description of the exemplary embodiments is intended to be read in connection with the accompanying drawings, which are to be considered part of the entire written description. It should be understood, however, that the present disclosure is not intended to be limited to the particular forms disclosed and that the drawings are not necessarily shown to scale. Rather, the present disclosure covers all modifications, equivalents, and alternatives that fall within the spirit and scope of these exemplary embodiments. In the description, relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The terms “couple,” “coupled,” “operatively coupled,” “operatively connected,” and the like should be broadly understood to refer to connecting devices or components together either mechanically, or otherwise, such that the connection allows the pertinent devices or components to operate with each other as intended by virtue of that relationship.

The present disclosure includes various embodiments of a medical device shaft that is configured to provide variable stiffness along the length of the shaft. Exemplary aspects of the medical device shaft include two or more zones of flexibility facilitated by one or more elongated members that are disposed at varying lengths from or neat to the proximal end of the shaft towards the distal end of the shaft. The variable stiffness shafts disclosed herein overcome the disadvantages of conventional medical device shafts and improve patient comfort during a procedure.

The medical device shafts disclosed herein may be used and adapted for a variety of different medical devices and procedures. For example, the shafts disclosed herein may be used on an endoscope, a cystoscope, a nephroscope, a bronchoscope, an arthroscope, a colonoscope, a laparoscope, a ureteroscope, a duodenoscope, a urethroscope, a catheter, etc., just to provide some non-limiting examples. These shafts may also be scaled based on the patient's characteristics. For example, the shafts may be configured based on the type of patient (e.g., human or animal), the patient's age (e.g., pediatric or adult), and/or the patient's size (e.g., small, medium, large, etc.), just to provide a few non-limiting examples.

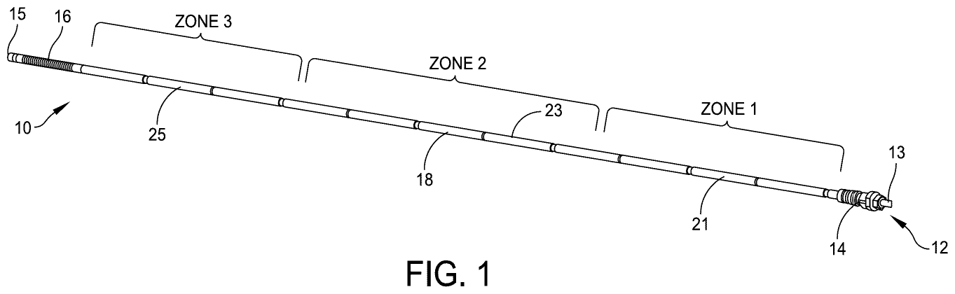

Referring now to the figures, FIG. 1 illustrates an isometric view of a medical device shaft 10 in accordance with some embodiments. The shaft 10 may define a void 12 along the length of the shaft 10. The void 12 may include one or more tubular cavities, channels, or lumens along the length of the shaft 10. The shaft 10 may also include a proximal end 13, a distal end 15, and a middle section 18 disposed between the proximal end 13 and the distal end 15. The proximal end 13 may include a handle 14 that is operatively coupled to another portion of the medical device that may provide some functionality, e.g., illumination, visual access, fluid supply, electrical supply, etc. The distal end 15 may include an operating member 16 configured to be inserted into a patient based on the type of medical procedure. For example, the operating member 16 may be the optics of an endoscope or an operating end of a catheter, just to provide a few non-limiting examples.

The middle section 18 of the shaft 10 may include two or more zones of stiffness along the length of the shaft 10. For example, the middle section 18 may include a first zone 21, a second zone 23, and a third zone 25. The first zone 21 may be disposed between the handle 14 and the second zone 23. The second zone 23 may be disposed between the first zone 21 and the third zone 25. The third zone 25 may be disposed between the operating member 16 and the second zone 23. The length of the zones (i.e., first zone 21, second zone 23, and third zone 25) may be dependent on the type of procedure used, characteristics of the patient, medical device used, or any combination thereof. For example, the third zone 25 may have a length between 25-55 millimeters in order to fit within a specific patient anatomy.

The shaft 10 further includes one or more elongated members disposed within the void 12 as will be discussed in further detail below. The shaft may also include one or more internal components of a medical device, such as optics wiring for an endoscope or tube for a catheter. The shaft 10 may be made of any medical grade materials, such as, for example, a polymeric tube (e.g., polyamide (PA), polyether block amide, polyurethane (PU), polyvinyl chloride (PVC), silicone, thermoplastic elastomers, etc.) with reinforced metal (e.g., stainless steel, etc.) or polymeric (e.g., polyether ether ketone (PEEK), etc.) braid. One of ordinary skill in the art will appreciate different types of materials useful for medical device shafts.

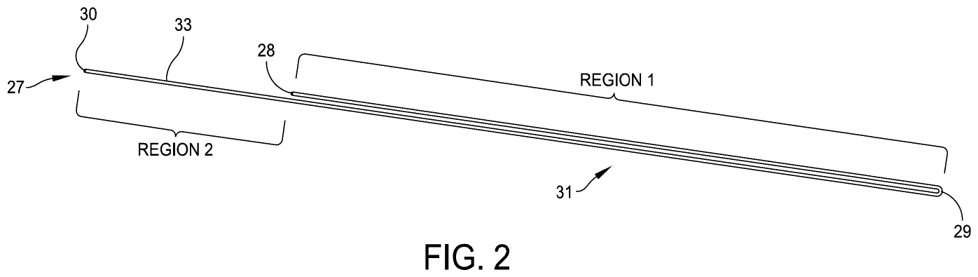

FIG. 2 illustrates an isometric view of a first example of an elongated member 27 of a medical device in accordance with some embodiments. The elongated member 27 may comprise one or more wires that are configured to be disposed within the void 12 of shaft 10. The elongated member 27 may include a first end 28, a second end 29, and a third end 30. The elongated member 27 may also include two or more regions of wire. For example, elongated member 27 may include a first region 31 and a second region 33. The elongated member 27 may be configured to be secured within shaft 10 and/or attached to the handle 14 with a press fit or male/female connection. For example, the second end 29 may be configured to be received within a female connection disposed within the shaft 10 and/or handle 14. In some embodiments, the shaft 10 has a separate lumen sized to receive elongated member 27 so that the elongated member 27 does not contact any of the medical device internal components.

The first region 31 of elongated member 27 may be disposed between the second end 29 and the second region 33. In the embodiment illustrated in FIG. 2, the elongated member 27 is a single wire that wraps back around at the second end 29 to provide two lengths of the wire within the first region 31. The second region 33 of elongated member 27 may be disposed between the first region 31 and the third end 30. The first region 31 is configured to align with the first zone 21 of the shaft 10 and the second region 33 is configured to align with the second zone 23 of the shaft 10. The two lengths of wire within the first zone 21 provide a stiffer section of shaft 10 than the second zone 23 and the third zone 25, thus providing sufficient push-ability of the shaft 10 by a user (e.g., physician, veterinarian, etc.). The single length of wire within the second zone 23 allows for a more flexible section of shaft 10, providing enough stiffness for push-ability of shaft 10 and also enough flexibility to navigate through a patient's anatomy. The third zone 25 of shaft 10 does not have any of elongated member 27 and thus provides the most flexible zone of three in order to allow sufficient flexibility to navigate through a patient's anatomy.

The elongated member 27 may be formed from metal (e.g., steel, stainless steel, copper, aluminum, some alloy, etc.), one or more polymeric materials (e.g., polyether ether ketone (PEEK), polyethylene (PE), polyamide (PA), etc.), reinforced structures such as glass-filled nylon, carbon fiber, etc., or may be formed from a composite material, such as, for example, a combination of metal, polymer, and/or some other material. The elongated member 27 may be smooth to not interfere with or damage other internal components disposed within shaft 10. In some embodiments, elongated member 27 may be ridged to provide a frictional force which, among other characteristics, may prevent elongated member 27 from moving within shaft 10. The diameter of elongated member 27 may be 15-50 thousandths of an inch according to some embodiments. However, in some embodiments of the present disclosure the elongated member 27 has a diameter of 26-35 thousandths of an inch. The diameter of the shaft 10 may be 2-15 times larger than the diameter of the elongated member 27.

Although elongated member 27 is illustrated as a single member, it is contemplated that elongated member 27 may also be comprised of one or more members that are coupled or fused together to make up one member. For example, elongated member 27 may be comprised of a first member made of metallic materials (e.g., steel, stainless steel, copper, aluminum, etc.) and a second member made of a polymeric material (e.g., polyether ether ketone (PEEK), polyethylene (PE), polyamide (PA), etc.), reinforced structures such as glass-filled nylon, carbon fiber, or other metal that are fused together to form a singular fused member.

Although elongated member 27 has been discussed as providing variable stiffness to shaft 10 of FIG. 1 based on the two lengths (e.g., first region 31 and second region 33) alone, it will be appreciated that additional features may provide additional flexibility or stiffness. For example, elongated member 27 may have a first size cross-section in the first region 31 that either changes at the second region 33 to a second cross-section or tapers along the entire length of the elongated member 27.

As another example, in the case of two members making up elongated member 27 as discussed above, the first region 31 may have a first shaped cross-section (e.g., circular, square, triangular, etc.) and the second region 33 may have a second shaped cross-section (e.g., circular, square, triangular, etc.). In some embodiments, the shaped cross-section of the first member may be different than the shaped cross-section of the second member.

As discussed above, elongated member 27, when inserted in shaft 10, provides shaft 10 with two or more regions of variable stiffness. The length of the regions may be dependent on the type of procedure used, characteristics of the patient, medical device used, or any combination thereof. For example, an endoscope may require a first region 31 that is two times the length of the second region 33. As another example, a catheter may require a first region 31 that is the same length as the second region 33. One of ordinary skill in the art with appreciate other applicable ratios between the first region 31 and the second region 33.

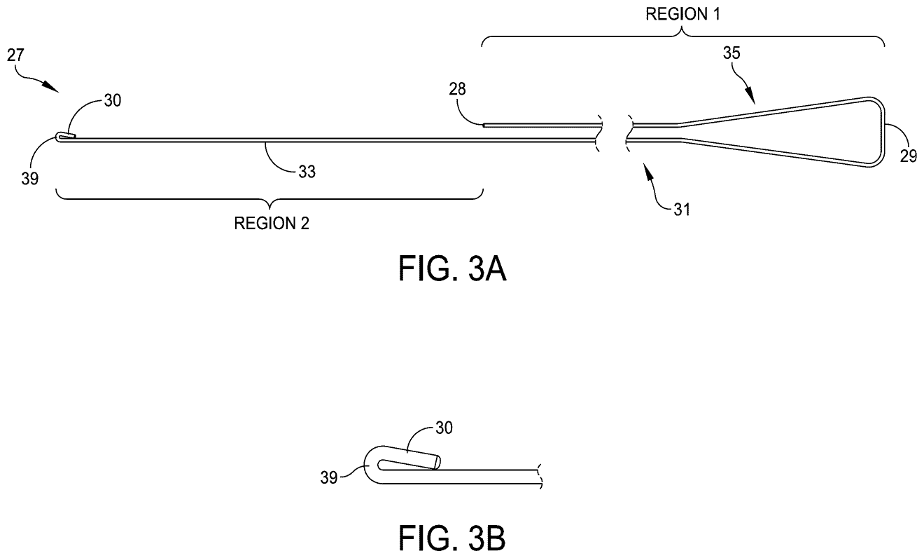

FIG. 3A illustrates a side view of a second example of an elongated member 27 of a medical device in accordance with some embodiments. Although FIG. 2 illustrates the second end 29 as being a loop configured to be mated with a respective female attachment within shaft 10 and/or handle 14, other shapes are contemplated. For example, FIG. 3A illustrates a generally triangular shape 35 for the second end 29 that is configured to mate to a respective female attachment within the shaft 10 and/or the handle 14. The generally triangular shape 35 of the second end 29 provides additional stability within the first region 31 compared to a simple loop as illustrated in FIG. 2. The generally triangular shape 35 may also be used to ensure the elongated member 27 does not migrate into shaft 10. It will be appreciated that other shapes for the second end 29 are contemplated.

FIG. 3B illustrates a side view of an end 30 of the second example of the elongated member 27 of a medical device in accordance with some embodiments. In some embodiments, the third end 30 of elongated member 27 has an end feature 39. The end feature 39 may be used to protect the shaft 10 and/or other internal components disposed within the shaft 10. As illustrated in FIG. 3B, the end feature 39 may be the third end 30 folded over on itself so as to form a round end feature 39. In some embodiments, the end feature 39 may include an attachable or integral dull portion to protect the shaft 10 and other internal components within the shaft 10. For example, the end feature 39 may be tapered, round, shaped, etc. so that the third end 30 is sufficiently dull. The end feature 39 may also be an attachable part that is configured to be attached to the third end 30. Although end feature 39 has been discussed with reference to the third end 30, it will be appreciated that first end 28 may also include a similar or different end feature 39 as discussed herein.

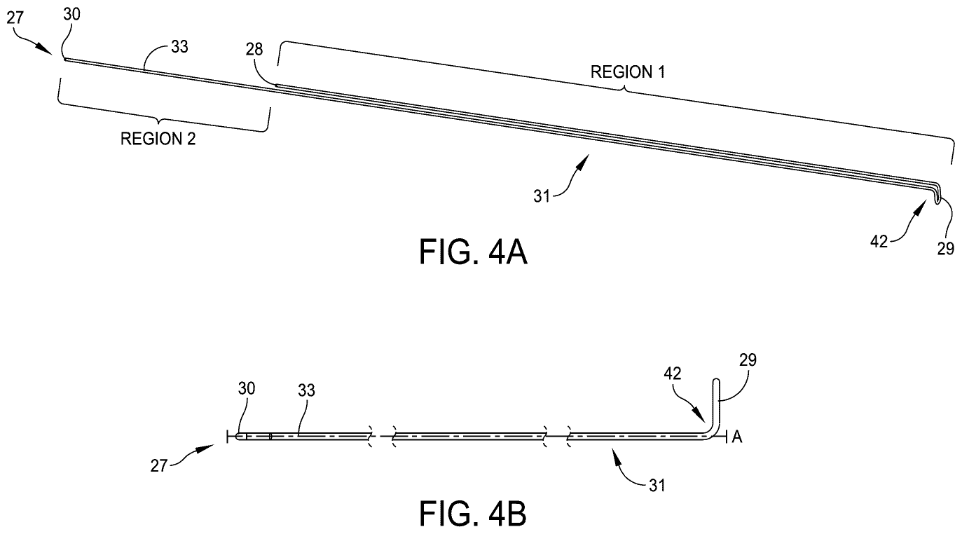

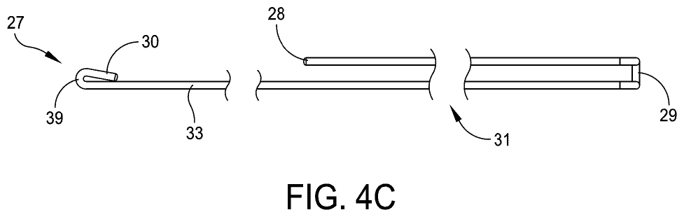

FIGS. 4A-4C illustrate an isometric, side, and top down view, respectively, of a third example of an elongated member 27 of a medical device in accordance with some embodiments. Although FIG. 2 illustrates the second end 29 as being a loop configured to be mated with a respective female attachment within shaft 10 and/or handle 14, other shapes and angles are contemplated. For example, FIGS. 4A-4C illustrate that the second end 29 may be disposed at an angle 42 from a longitudinal axis A of the elongated member 27. The angle 42 may be used to facilitate mating to a respective female attachment within the shaft 10 and/or the handle 14. The angle 42 may be anywhere between 1-179 degrees from the longitudinal axis A of elongated member 27. As an example, the angle 42 of the second end 29 may be approximately 90 degrees from the longitudinal axis A according to some embodiments. In other embodiments, the angle 42 may be, for example, in the range of 85-95 degrees, 80-100 degrees, or 70-110 degrees from the longitudinal axis A, and all subranges therein.

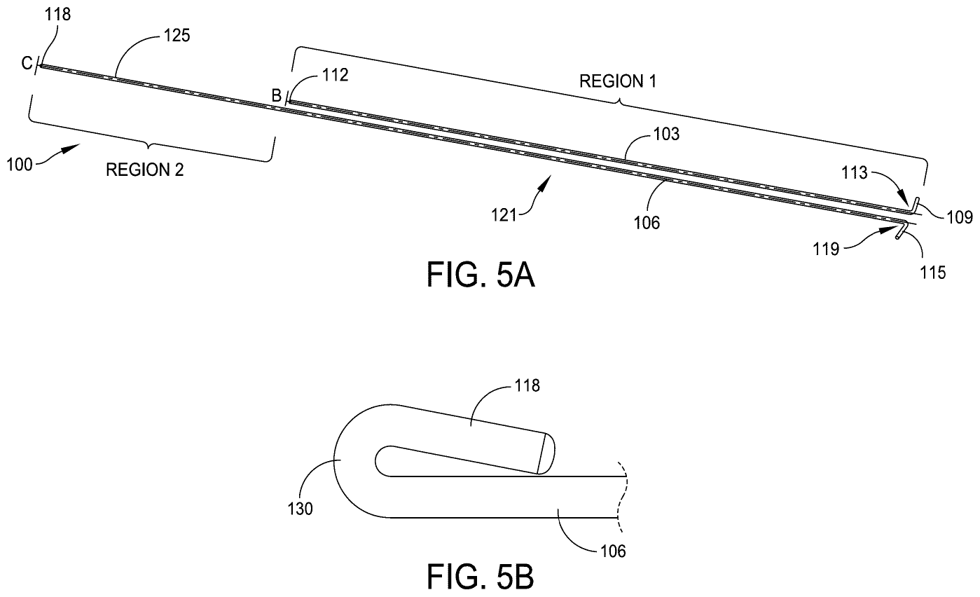

FIG. 5A illustrates an isometric view of a fourth example of an elongated member 100 of a medical device in accordance with some embodiments. The elongated member 100 may comprise two or more wires that are configured to be disposed within the one or more lumens of the void 12 within the shaft 10 of FIG. 1. As an example, the two or more wires may be disposed within a singular lumen together or may be disposed within separate lumens within the void 12. The elongated member 100 may include a first wire 103 and a second wire 106. The first wire 103 may include a first end 109 and a second end 112. The first end 109 of the first wire 103 may be disposed at an angle 113 from a longitudinal axis B of the first wire 103. The second wire 106 may include a first end 115 and a second end 118. The first end 115 of the second wire 106 may also be disposed at an angle 119 from a longitudinal axis C of the second wire 106.

The elongated member 100 may be configured to be secured within the shaft 10 and/or attached to the handle 14 with, for example, a press fit or male/female connection. For example, the first end 109 of the first wire 103 and the first end 115 of the second wire 106 may be configured to be received within one or more female connections disposed within one or more lumens of the void 12 and/or attached to the handle 14. In some embodiments, the angle 113 of the first end 109 of the first wire 103 and the angle 119 of the first end 115 of the second wire 106 may be used to facilitate mating to one or more respective female attachments. The angles 113, 119 may be anywhere between 1-179 degrees from the longitudinal axes B, C of each of the first wire 103 and the second wire 106 respectively. As an example, the angles 113, 119 may be approximately 90 degrees from the longitudinal axes B and C respectively according to some embodiments. In other embodiments, the angles 113, 119 may be, for example, in the range of 85-95 degrees, 80-100 degrees, or 70-110 degrees from the longitudinal axes B and C respectively, and all subranges therein.

The elongated member 100 may define two or more regions of wire. For example, elongated member 100 may define a first region 121 and a second region 125. The first region 121 of elongated member 100 may be disposed between the first ends 109, 115 and the second region 125. The second region 125 of elongated member 100 may be disposed between the first region 121 and the second end 118 of the second wire 106. The first region 121 is configured to align with the first zone 21 of the shaft 10 and the second region 125 is configured to align with the second zone 23 of the shaft 10. The two wires of elongated member 100 within the first zone 21 provide a stiffer section of shaft 10 than the second zone 23 and the third zone 25, thus providing sufficient push-ability of the shaft 10 by a user (e.g., physician). The single length of wire of the elongated member 100 within the second zone 23 allows for a more flexible section of shaft 10, providing enough stiffness for push-ability of shaft 10 and also enough flexibility to navigate through a patient's anatomy. The third zone 25 of shaft 10 does not have either wire 103, 106 of elongated member 27 and thus provides the most flexible zone of the three in order to allow sufficient flexibility to navigate through a patient's anatomy.

The elongated member 100 may be formed from metal (e.g., steel, stainless steel, copper, aluminum, some alloy, etc.), one or more polymeric materials (e.g., polyether ether ketone (PEEK), polyethylene (PE), polyamide (PA), etc.), reinforced structures such as glass-filled nylon, carbon fiber, etc., or may be formed from a composite material, such as, for example, a combination of metal, polymer, and/or some other material. The elongated member 100 may be smooth to not interfere with or damage other internal components disposed within shaft 10. In some embodiments, elongated member 100 may be ridged to provide a frictional force, which, among other characteristics, may prevent elongated member 100 from moving within shaft 10. The diameter of elongated member 100 may be 15-50 thousandths of an inch according to some embodiments. However, in some embodiments of the present disclosure the elongated member 100 has a diameter of 26-35 thousandths of an inch. The diameter of the shaft 10 may be 2-15 times larger than the diameter of the elongated member 100.

In some embodiments, the first wire 103 and the second wire 106 may have different diameters to change the stiffness either the first region 121 or the second region 125. For example, the first wire 103 may have a diameter that is thicker than the diameter of the second wire 106 to increase the proximal end 13 rigidity within the first zone 21 of the shaft 10.

Although each of the first wire 103 and the second wire 106 are illustrated as a single member, it is contemplated that elongated member 100 may also be comprised of one or more members that are coupled or fused together to make up one member. For example, the first wire 103 and/or the second wire 106 may be comprised of a first member made of metallic materials (e.g., steel, stainless steel, copper, aluminum, etc.) and a second member made of a polymeric material (e.g., polyether ether ketone (PEEK), polyethylene (PE), polyamide (PA), etc.), reinforced structures such as glass-filled nylon, carbon fiber, or other metal that are fused together to form a singular fused member.

Although elongated member 100 has been discussed as providing variable stiffness to shaft 10 of FIG. 1 based on the two lengths of the first wire 103 and the second wire 106 within the first region 121 and second region 125 alone, it will be appreciated that additional features may provide additional flexibility or stiffness. For example, elongated member 100 may have a first size cross-section in the first region 121 that either changes at the second region 125 to a second size cross-section or tapers along the entire length of the elongated member 100.

As another example, in the case of two members making up the second wire 106 as discussed above, the first region 121 may have a first shaped cross-section (e.g., circular, square, triangular, etc.) and the second region 125 may have a second shaped cross-section (e.g., circular, square, triangular, etc.). In some embodiments, the shaped cross-section of the first member may be different than the shaped cross-section of the second member.

As discussed above, elongated member 100, when inserted in shaft 10, provides shaft 10 with two or more regions of variable stiffness. The length of the regions may be dependent on the type of procedure used, characteristics of the patient, medical device used, or any combination thereof. For example, an endoscope may require a first region 121 that is two times the length of the second region 125. As another example, a catheter may require a first region 121 that is the same length as the second region 125. One of ordinary skill in the art with appreciate other applicable ratios between the first region 121 and the second region 125.

FIG. 5B illustrates a side view of an end 118 of the fourth example of the elongated member 100 of a medical device in accordance with some embodiments. In some embodiments, the second end 118 of the second wire 106 has an end feature 130. The end feature 130 may be used to protect the shaft 10 and/or other internal components disposed within the shaft 10. As illustrated in FIG. 5B, the end feature 130 may be the second end 118 of the second wire 106 folded over on itself so as to form a round end feature 130. In some embodiments, the end feature 130 may include an attachable or integral dull portion to protect the shaft 10 and other internal components within the shaft 10. For example, the end feature 130 may be tapered, round, shaped, etc. so that the second end 118 is sufficiently dull. The end feature 130 may also be an attachable part that is configured to be attached to the second end 118 of the second wire 106. Although end feature 130 has been discussed with reference to the second end 118 of the second wire 106, it will be appreciated that first end 109 of the first wire 103, the second end 112 of the first wire 103, and the first end 115 of the second wire 106 may also include a similar or different end feature 130 as discussed herein.

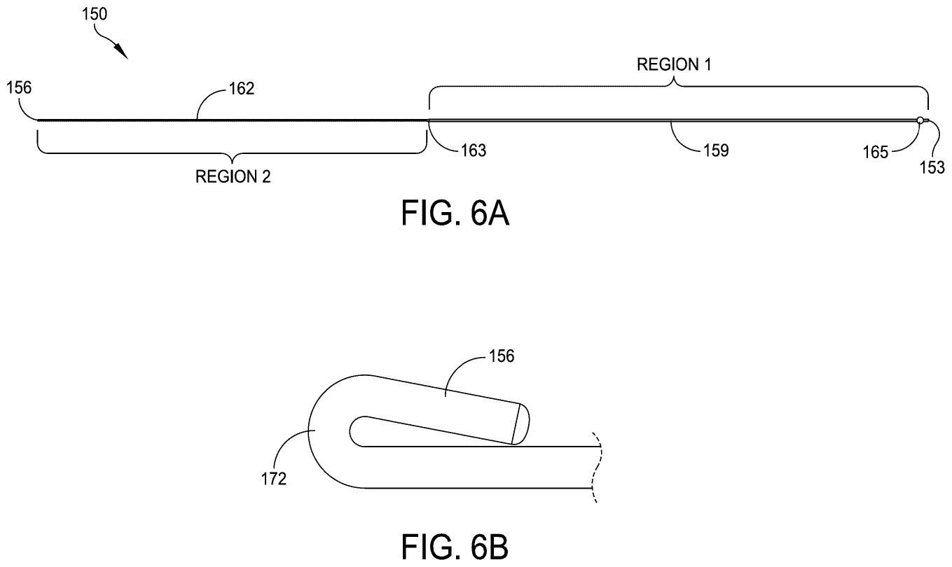

FIG. 6A illustrates a side view of a fifth example of an elongated member 150 of a medical device in accordance with some embodiments. The elongated member 150 may comprise one or more wires that are configured to be disposed within one or more lumens of the void 12 of shaft 10. The elongated member 150 may include a first end 153 and a second end 156. The elongated member 150 may also include two or more regions of wire. For example, elongated member 150 may include a first region 159 and a second region 162 that is divided by a transition 163. The first end 153 may include a stamp 165.

The elongated member 150 may be configured to be secured within shaft 10 and/or attached to the handle 14 with a press fit or male/female connection. For example, the stamp 165 may be configured to be received within a female connection disposed within the shaft 10 and/or handle 14. In other embodiments, the elongated member 150 has a formed first end 153 configured to be secured within the shaft 10 and/or attached to the handle, similar to elongated members 27 and 100. In some embodiments, the shaft 10 has a separate lumen sized to receive elongated member 150 so that the elongated member 150 does not contact any of the medical device internal components.

The first region 159 of elongated member 150 may extend between the first end 153 and the second region 162. In the embodiment illustrated in FIG. 6A, the elongated member 150 is a single wire that tapers at the transition 163 from a larger diameter to a smaller diameter. The second region 162 of elongated member 150 may extend between the first region 159 and the second end 156. The first region 159 is configured to align with the first zone 21 of the shaft 10 and the second region 162 is configured to align with the second zone 23 of the shaft 10. The thicker portion of elongated member 150 within the first zone 21 provides a stiffer section of shaft 10 than the second zone 23 and the third zone 25, thus providing sufficient push-ability of the shaft 10 by a user (e.g., physician, veterinarian, etc.). The smaller diameter portion of elongated member 150 within the second zone 23 allows for a more flexible section of shaft 10, providing enough stiffness for push-ability of shaft 10 and also enough flexibility to navigate through a patient's anatomy. The third zone 25 of shaft 10 does not have any of elongated member 150 and thus provides the most flexible zones of three in order to allow sufficient flexibility to navigate through a patient's anatomy.

The elongated member 150 may be formed from metal (e.g., steel, stainless steel, copper, aluminum, some alloy, etc.), one or more polymeric materials (e.g., polyether ether ketone (PEEK), polyethylene (PE), polyamide (PA), etc.), reinforced structures such as glass-filled nylon, carbon fiber, etc., or may be formed from a composite material, such as, for example, a combination of metal, polymer, and/or some other material. The elongated member 150 may be smooth to not interfere with or damage other internal components disposed within shaft 10. In some embodiments, elongated member 150 may be ridged to provide a frictional force which, among other characteristics, may prevent elongated member 150 from moving within shaft 10. The diameters of the various portions elongated member 150 may be 10-75 thousandths of an inch according to some embodiments. However, in some embodiments of the present disclosure the elongated member 150 has a diameter of 40-75 thousandths of an inch for the first region 159 and 26-35 thousandths of an inch for the second region 162. The diameter of the shaft 10 may be 2-15 times larger than the diameter of either region 159, 162 of the elongated member 150.

Although elongated member 150 is illustrated as a single member, it is contemplated that elongated member 150 may also be comprised of one or more members that are coupled or fused together to make up one member. For example, elongated member 150 may be comprised of a first member made of metallic materials (e.g., steel, stainless steel, copper, aluminum, etc.) and a second member made of a polymeric material (e.g., polyether ether ketone (PEEK), polyethylene (PE), polyamide (PA), etc.), reinforced structures such as glass-filled nylon, carbon fiber, or other metal that are fused together to form a singular fused member.

Although elongated member 150 has been discussed as providing variable stiffness to shaft 10 of FIG. 1 based on the change in diameters for the two regions 159, 162 alone, it will be appreciated that additional features may provide additional flexibility or stiffness. For example, elongated member 150 may have a cross-section that tapers along the entire length of the elongated member 150.

As another example, in the case of two members making up elongated member 150 as discussed above, the first region 159 may have a first shaped cross-section (e.g., circular, square, triangular, etc.) and the second region 162 may have a second shaped cross-section (e.g., circular, square, triangular, etc.). In some embodiments, the shaped cross-section of the first member may be different than the shaped cross-section of the second member.

As discussed above, elongated member 150, when inserted in shaft 10, provides shaft 10 with two or more regions of variable stiffness. The length of the regions may be dependent on the type of procedure used, characteristics of the patient, medical device used, or any combination thereof. For example, an endoscope may require a first region 159 that is two times the length of the second region 162. As another example, a catheter may require a first region 159 that is the same length as the second region 162. One of ordinary skill in the art will appreciate other applicable ratios between the first region 159 and the second region 162.

FIG. 6B illustrates a side view of an end 156 of the fifth example of the elongated member 150 of a medical device in accordance with some embodiments. In some embodiments, the second end 156 of the elongated member 150 has an end feature 172. The end feature 172 may be used to protect the shaft 10 and/or other internal components disposed within the shaft 10. As illustrated in FIG. 6B, the end feature 172 may be the second end 156 of the elongated member 150 folded over on itself so as to form a round end feature 172. In some embodiments, the end feature 172 may include an attachable or integral dull portion to protect the shaft 10 and other internal components within the shaft 10. For example, the end feature 172 may be tapered, round, shaped, etc. so that the second end 156 is sufficiently dull. The end feature 172 may also be an attachable part that is configured to be attached to the second end 156 of the elongated member. Although end feature 172 has been discussed with reference to the second end 156 of the elongated member 150 it will be appreciated that first end 153 may also include a similar or different end feature 172 as discussed herein.

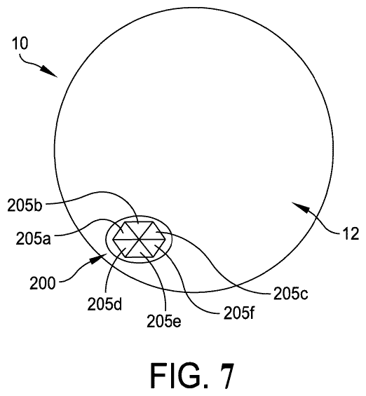

FIG. 7 illustrates a cross sectional view of a shaft 10 having a sixth example of the elongated member 200 of a medical device in accordance with some embodiments. The elongated member 200 may be disposed within the one or more lumens of the void 12. The elongated member 200 may include a plurality of smaller rods 205a-f that are configured to be inserted and removed from the shaft to adjust the needed stiffness depending on the type of procedure or patient. For example, a user may determine that a shaft may need more flexibility for a particular procedure and remove one or more rods 205a-f to achieve the desired stiffness of the shaft 10.

In some embodiments, the rods 205a-f may be different in size, shape, length, material, stiffness, or any combination thereof. As an example, rods 205a-c may be a first length so as to be disposed along the length of the first zone 21 and the second zone 23 of the shaft 10, and rods 205d-f may be a second length so as to be disposed along only the first zone 21 of the shaft 10. In this example, the shaft 10 is configured for variable stiffness along the two or more zones as described above regarding elongated members 27 and 100.

It may be emphasized that the above-described embodiments, particularly any “preferred” embodiments, are merely possible examples of implementations, set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

While this specification contains many specifics, these should not be construed as limitations on the scope of any disclosures, but rather as descriptions of features that may be specific to a particular embodiment. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. A system comprising:
a shaft defining a void and having a length extending between a proximal end and a distal end; and
an elongated member disposed within the void of the shaft and along at least a portion of the length of the shaft, wherein the elongated member is configured to define at least two regions, wherein the at least two regions each have a different stiffness, facilitating a variable stiffness along the length of the shaft.

2. The system of claim 1, wherein the void of the shaft includes one or more lumens along the length of the shaft.

3. The system of claim 2, wherein the elongated member is disposed in one of the one or more lumens.

4. The system of claim 1, wherein the elongated member comprises a wire that wraps back around at an end of the wire such that there are two lengths of wire within a first region of the at least two regions and one length of wire within a second region of the at least two regions.

5. The system of claim 4, wherein the shaft defines a first zone, a second zone, and a third zone, and wherein the first region of the wire is disposed within the first zone, the second region of the wire is disposed within the second zone, and the third zone does not include any length of the wire.

6. The system of claim 4, wherein the end of the wire that wraps back around is disposed at an angle from a longitudinal axis of the shaft.

7. The system of claim 1, wherein the elongated member comprises a first wire and a second wire, the first wire being shorter than the second wire.

8. The system of claim 7, wherein the first wire and the second wire are disposed in a first region of the at least two regions and the second wire is disposed in a second region of the at least two regions.

9. The system of claim 8, wherein the shaft defines a first zone, a second zone, and a third zone, and wherein the first region is disposed within the first zone, the second region is disposed within the second zone, and the third zone does not include any length of the first wire or the second wire.

10. The system of claim 7, wherein the first wire has a first size cross-section and the second wire has a second size cross-section.

11. The system of claim 7, wherein the first wire and the second wire each extend between a first end a second end, the first ends of the first wire and the second wire each defining an angle and the second ends of the first wire and the second wire each defining an end feature.

12. A system comprising:
a shaft defining a void and having a length extending between a proximal end and a distal end; and
an elongated member disposed within the void of the shaft and along at least a portion of the length of the shaft, wherein the elongated member has a first diameter that tapers to at least a second diameter along a length of the elongated member, facilitating a variable stiffness along the length of the shaft.

13. The system of claim 12, wherein the void of the shaft includes one or more lumens along the length of the shaft.

14. The system of claim 13, wherein the elongated member is disposed in one of the one or more lumens.

15. The system of claim 12, wherein the elongated member comprises one or more wires.

16. The system of claim 12, wherein the shaft defines a first zone, a second zone, and a third zone, and wherein the elongated member tapers along the length of the elongated member such that the first zone is stiffer than the second zone and the third zone.

17. A system comprising:
a shaft defining a void and having a length extending between a proximal end and a distal end; and
a plurality of removable rods disposed within the void of the shaft, wherein a first portion of the plurality of removable rods are disposed at a length that is shorter than a second portion of the plurality of removable rods, facilitating a variable stiffness along the length of the shaft.

18. The system of claim 17, wherein the void of the shaft includes one or more lumens along the length of the shaft.

19. The system of claim 18, wherein the plurality of removable rods are disposed in one of the one or more lumens.

20. The system of claim 17, wherein the shaft defines a first zone, a second zone, and a third zone, and wherein the first portion and the second portion of the plurality of removable rods are disposed within the first zone, the second portion of the plurality of removable rods are disposed within the second zone, and the third zone of the shaft does not include any of the plurality of removable rods.
